(19) United States
(12) Patent Application Publication
(10) Pub. No.: US 2013/0064850 A1
(43) Pub. Date: Mar. 14, 2013

(54) Using Modified Plasmids to Suppress Antibiotic Resistant Pathogens

(71) Applicant: Kevin Roe, San Jose, CA (US)

(72) Inventor: Kevin Roe, San Jose, CA (US)

(21) Appl. No.: 13/341,281

(22) Filed: Dec. 30, 2011

Related U.S. Application Data
(60) Provisional application No. 61/524,281, filed on Aug. 16, 2011.

(51) Int. Cl. A61K 39/02; A61P 31/04; A61K 35/74; A61P 37/04; C12N 15/63; A61K 35/66

(52) U.S. Cl. 424/234.1; 435/440; 424/93.2

(57) ABSTRACT

Methods of suppressing at least one species of micro-organism that is pathological to an organism. A first embodiment includes a method of suppressing at least one species of micro-organism that is pathological to an organism. A second embodiment includes a method of suppressing at least one species of micro-organism that is pathological to a mammal. A third embodiment includes a method of suppressing at least one species of bacteria that is pathological to a mammal.

### CROSS-REFERENCE TO RELATED APPLICATIONS

This application claims priority from a U.S. provisional patent application Ser. No. 61/524,281, filed Aug. 16, 2011, by the same inventor, entitled “Using Modified Plasmids to Suppress Antibiotic Resistant Pathogens,” which is hereby incorporated by reference.

### BACKGROUND OF THE INVENTION

1. Field of the Invention

This invention relates generally to an anti-pathogen technique, and more specifically to modifying plasmids in order to use modified plasmids in one type of micro-organism to suppress another type of micro-organism.

2. Description of the Prior Art

Extended-spectrum beta-lactamase producing bacteria have become increasingly prevalent, now causing a large percentage of all fatal infections in the U.S. and in the rest of the world. These bacteria are not only resistant to virtually all beta-lactam antibiotics, but also resistant to many of the other major classes of antibiotics, including the carbapenems, fluoroquinolones, aminoglycosides, tetracyclines, macrolides, lincosamides, glycopeptides, sulfonamides, vancomycin, and chloramphenicol. The following reference provides further background information concerning this matter and is incorporated by reference—Funnell, B. E. & Phillips, G. J., editors. Plasmid Biology. 473-491 (American Society for Microbiology, Washington, D.C., 2004).

Antibiotic resistance is usually transmitted between bacterial species and/or genus by plasmids, which can essentially act as a large bacterial lending library of external chromosomes for enzyme functions beyond the normal chromosomally encoded repertoire of individual bacterial species. Plasmids in general can encode an enormous variety of functions which are not essential to survival of the host or plasmid, but which encode extra functions that extend the host's range by increasing the host's fitness in atypical environments. The following reference provides further background information concerning this matter and is incorporated by reference—Summers, D. K. The Biology of Plasmids. 1-11 (Blackwell Science Ltd. Oxford, U.K. 1996). Plasmids can protect a host against heavy metals, ionizing radiation, and even other bacteria (by encoding bioreactive compounds such as colicins and antibiotics to kill other bacteria lacking the plasmid). Plasmids can also increase the virulence and pathogenicity of host bacteria by encoding new toxins and various other bacterial colonization aids. The following reference provides further background information concerning this matter and is incorporated by reference—Gordon, D. M. The potential of bacteriocin-producing probiotics and associated caveats. Future Microbiology 4, 941-943 (2009).

Plasmids can either be circular or linear. They can exist in Gram-negative and Gram-positive bacteria. The three critical properties of plasmids are copy number, host-dependence and range, and response to environmental conditions. Plasmids vary in size from a few hundred base pairs to hundreds of thousands of base pairs. Plasmids vary in their copy number within a cell from 1 to 30, in their host range from one to several species, and in the new environmental traits they encode. The following reference provides further background information concerning this matter and is incorporated by reference—Espinosa, M. et al. Plasmid Replication and Copy Number Control, The Horizontal Gene Pool Bacterial Plasmids and Gene Spread. 1 (Harwood Academic Publishers, Amsterdam, the Netherlands, 2000).

Plasmids can be transferred by transduction and transformation. The following reference provides further background information concerning this matter and is incorporated by reference—Hayes, F. E. coli Plasmid Vectors Methods and Applications. (ed. Casali, N., & Preston, A.) 1-18 (Humana Press, Totowa, N.J., 2003). But the most common method of transfer of DNA from a donor bacterium to a recipient bacterium uses a pili or conjugation apparatus, which is called conjugation, and even genetic transfers between unrelated genera of bacteria are common, even between Gram-negative and Gram-positive bacteria. The following reference provides further background information concerning this matter and is incorporated by reference—Trieu-Cuot, P., Derlot, E., & Courvalin, P. Enhanced conjugative transfer of plasmid DNA from Escherichia coli to Staphylococcus aureus and Listeria moncytogenes. FEMS Microbiol. Lett. 109, 19-23 (1993). Plasmid conjugation between bacteria and fungi is also feasible and has been observed. The following reference provides further background information concerning this matter and is incorporated by reference—Bates, S., Cashmore, A. M., & Wilkins, B. M. IncP plasmids are unusually effective in mediating conjugation of Escherichia coli and Saccharomyces cerevisiae: involvement of the Tra2 mating system. J. Bacteriol. 180, 6538-6543 (1998).

More than 25 different groups of plasmids have been defined on the basis of incompatibility, and each has a distinct conjugation system. If two plasmids are members of the same incompatibility group, the introduction of one of the two plasmids by conjugation, transformation, or transduction into a bacterial cell carrying the other plasmid destabilizes the inheritance of the previous plasmid. Some plasmids are self-transmissible plasmids, such as members of the P, N, and W incompatibility groups. The following reference provides further background information concerning this matter and is incorporated by reference—Guiney, D. G. Broad Host Range Conjugative and Mobilizable Plasmids in Gram-Negative Bacteria. Bacterial Conjugation 75-103 (Plenum Press, New York, N.Y., 1993).

For example, plasmids can be transferred by conjugation after release of peptide sex pheromone by recipient cells to trigger donor cells to produce surface components to induce cell clumping between bacterial members of the Enterococcus. The following reference provides further background information concerning this matter and is incorporated by reference—Dunny, G. M., & Leonard, B. A. Cell-cell communication in gram-positive bacteria. Annual Review Microbiology 51, 527-564 (1997). The plasmid transfer frequency even for common, non-modified plasmids can be very high (approximately 10 exp −2 or higher). A second class of transfer uses conjugative transposons, but the transfer frequency is quite low (approximately 10 exp −6). A third class of transfer uses the broad host range conjugative plasmids. The natural transfer frequency for commonly occurring non-modified plasmids varies widely (10 exp −6 to 10 exp −3). The following reference provides further background information concerning these matters and is incorporated by reference—Macrina, F. L., & Archer G. L. Conjugation and Broad Host Range Plasmids in Streptococci and Staphylococci, Bacterial Conjugation 313-329 (Plenum Press, New York, N.Y., 1993). Conjugative plasmid transfer can be maximized with a high donor cell density, and a proper ratio of donor cells to recipient cells, and the transfer frequency can be high (10 exp-1) even for non-modified, commonly occurring plasmids for typical pathogenic bacteria like Staphylococcus aureus, over a broad range of pH and at temperatures ranging from 25 degrees C. to 37 degrees C. The following reference provides further background information concerning these matters and is incorporated by reference—Al-Masaudi, S. B., Russell, A. D., Day, M. J. Factors affecting conjugative transfer of plasmid pWG613, determining gentamicin resistance in Staphylococcus aureus. J. Med. Microbiology, 34, 103-107 (1991). Conjugative plasmid transfer rates could be further maximized by use of modified plasmids that are specifically optimized for maximum transfer rates to targeted recipient pathogens.

The plasmid genes and sequences for plasmid replication and control are contained in a small region called the basic replicon. The basic replicon includes the (ori), the origin of replication, the (cop/inc genes) for the control of the initiation of replication, and the (rep genes) for encoding the proteins for replication of the plasmid. The following reference provides further background information concerning these matters and is incorporated by reference—Espinosa, M. et al. Plasmid Replication and Copy Number Control. The Horizontal Gene Pool Bacterial Plasmids and Gene Spread. 1-18 (Harwood Academic Publishers, Amsterdam, the Netherlands, 2000).

Plasmids can carry insertion sequences that enable plasmids to become part of the bacterial chromosome. Plasmids also carry transposons that transfer themselves or a copy of themselves to DNA molecules. The main difference between insertion sequences and transposons is that transposons can insert one or more genes into the bacterial chromosome and these genes will then be expressed as a phenotype. For example, composite transposons comprise two identical insertion sequences flanking one or more genes, transposing the entire group into the bacterial chromosome and creating a phenotype expressing the one or more genes. The following reference provides further background information concerning these matters and is incorporated by reference—Merlin, C., Mahillon, J., Nesvera, J., Toussaint, A. Gene Recruiters and Transporter: the Modular Structure of Bacterial Mobile Elements. The Horizontal Gene Pool Bacterial Plasmids and Gene Spread. 363-409 (Harwood Academic Publishers, Amsterdam, the Netherlands, 2000).

Bacterial cells will eventually eliminate incompatible plasmids during successive generations, but they can continue to carry many plasmids, if they are compatible. For example, Enterobacteria can carry up to seven compatible plasmids. The following reference provides further background information concerning these matters and is incorporated by reference—Smalla, K., Osborn, A. M., Wellington, E. M. H. Isolation and Characterisation of Plasmids from Bacteria. The Horizontal Gene Pool Bacterial Plasmids and Gene Spread. 207-248 (Harwood Academic Publishers, Amsterdam, the Netherlands, 2000).

### SUMMARY OF THE INVENTION

A first aspect of the invention is directed to a method suppressing at least one species of micro-organism that is pathological to an organism, including: modifying at least one type of plasmid to produce at least one type of modified plasmid that will result in at least one anti-pathogen effect; inserting the at least one type of modified plasmid into donor micro-organisms; increasing the donor micro-organisms in number by incubation of the donor micro-organisms; extracting donor micro-organisms containing a plurality of modified plasmids; and packaging the donor micro-organisms containing the modified plasmids for a beneficial use.

A second aspect of the invention is directed to a method suppressing at least one species of micro-organism that is pathological to a mammal, including: modifying at least one type of plasmid to produce at least one anti-pathogen effect; inserting the at least one type of plasmid into donor micro-organisms; increasing donor micro-organisms in number by incubation of the donor micro-organisms; extracting donor micro-organisms containing a plurality of modified plasmids; and packaging the donor micro-organisms containing the modified plasmids for a beneficial use.

A third aspect of the invention is directed to a method suppressing at least one species of micro-organism that is pathological to a mammal, including: modifying at least one type of plasmid to produce at least one anti-pathogen effect; inserting the at least one type of plasmid into donor micro-organisms; increasing donor micro-organisms in number by incubation of the donor micro-organisms; extracting donor micro-organisms containing a plurality of modified plasmids; packaging the donor micro-organisms containing the modified plasmids for a beneficial use; introducing a plurality of donor micro-organisms into a mammal; and facilitating the transfer of the at least one type of plasmid from the plurality of donor micro-organisms to at least one type of host micro-organism in the mammal, to suppress at least one species of micro-organism that is pathological to the mammal.

### BRIEF DESCRIPTION OF THE DRAWINGS

FIG. 1 illustrates a flowchart of a method to use modified plasmids in donor micro-organisms for a beneficial use, in accordance with one embodiment of the invention.

FIG. 2 illustrates a flowchart of a method to manufacture modified plasmids in donor micro-organisms for a beneficial use, in accordance with one embodiment of the invention.

FIG. 3 illustrates a flowchart of a method to use modified plasmids in donor micro-organisms for a beneficial use, in accordance with another embodiment of the invention.

FIG. 4 illustrates a flowchart of a method to use modified plasmids in donor micro-organisms for a beneficial use, in accordance with another embodiment of the invention.

FIG. 5 illustrates a flowchart of a method to use modified plasmids in donor micro-organisms for a beneficial use, in accordance with another embodiment of the invention.

FIG. 6 illustrates a flowchart of a method to use modified plasmids in donor micro-organisms for a beneficial use, in accordance with another embodiment of the invention.

FIG. 7 illustrates a flowchart of a method to use modified plasmids in donor micro-organisms for a beneficial use, in accordance with another embodiment of the invention.

FIG. 8 illustrates a flowchart of a method to use modified plasmids in donor micro-organisms for a beneficial use, in accordance with another embodiment of the invention.

FIG. 9 illustrates a flowchart of a method to use modified plasmids in donor micro-organisms for a beneficial use, in accordance with another embodiment of the invention.

FIG. 10 illustrates a flowchart of a method to use modified plasmids in donor micro-organisms for a beneficial use, in accordance with another embodiment of the invention.

FIG. 11 illustrates a flowchart of a method to use modified plasmids in donor micro-organisms for a beneficial use, in accordance with another embodiment of the invention.

FIG. 12 illustrates a flowchart of a method to use modified plasmids in donor micro-organisms for a beneficial use, in accordance with another embodiment of the invention.

FIG. 13 illustrates a flowchart of a method to use modified plasmids in donor micro-organisms for a beneficial use, in accordance with another embodiment of the invention.

FIG. 14 illustrates a flowchart of a method to use modified plasmids in donor micro-organisms for a beneficial use, in accordance with another embodiment of the invention.

FIG. 15 illustrates a flowchart of a method to use modified plasmids in donor micro-organisms for a beneficial use, in accordance with another embodiment of the invention.

FIG. 16 illustrates a flowchart of a method to use modified plasmids in donor micro-organisms for a beneficial use, in accordance with another embodiment of the invention.

FIG. 17 illustrates a flowchart of a method to use modified plasmids in donor micro-organisms for a beneficial use, in accordance with another embodiment of the invention.

FIG. 18 illustrates a flowchart of a method to use modified plasmids in donor micro-organisms for a beneficial use, in accordance with another embodiment of the invention.

FIG. 19 illustrates a flowchart of a method to use modified plasmids in donor micro-organisms for a beneficial use, in accordance with another embodiment of the invention.

FIG. 20 illustrates a flowchart of a method to use modified plasmids in donor micro-organisms for a beneficial use, in accordance with another embodiment of the invention.

### DETAILED DESCRIPTION OF THE PREFERRED EMBODIMENTS

A plasmid that would be a potentially good candidate for modification would not be too small or too large in the base pair count and have a very broad range of micro-organisms that would easily acquire it and keep it. Such candidate plasmids for modification for use against specific pathogens can be identified from a vast scientific literature of studies on different plasmids of all types and sizes. These plasmids can be purchased in carrier micro-organisms from microbiology supply houses. Such plasmids can also frequently be alternatively acquired at little or no cost from various university microbiology research labs that have recently studied the plasmids. There are several techniques in which modified plasmids can be used as agents against pathogens. Five basic strategies for using modified plasmids to suppress pathogens are discussed below.

### Discussion

Plasmids have been successfully used to convert bacteria such as E. coli into vaccine factories for several years, or have been used to make antigen-based or DNA-based vaccines to confer humoral immunity to some future pathogen infection. The following reference provides further background information concerning these matters and is incorporated by reference—Gregoriadis, G. Genetic vaccines: strategies for optimization. Pharmaceutical Research 15, 661 (1998). But the use of modified plasmids, either carried by a donor cell or in a pure plasmid application, as a direct agent to immediately attack an existing pathogenic infection at first sight would not appear feasible, since plasmids typically help bacteria to extend their repertoire of capabilities for survival. For example, as noted above, antibiotic resistance is usually transmitted between bacterial species and/or genus by plasmids. In fact, bacteria such as the Enterococci and Staphylococci have even demonstrated the ability to resist antibiotics such as vancomycin by simultaneously activating an operon of multiple genes shuffled and separately reassembled from multiple plasmid sources. The following reference provides further background information concerning these matters and is incorporated by reference—Amabile-Cuevas, C. F. New antibiotics and new resistance. American Scientist. 91, 138 (2003).

However, there are several ways to use plasmids as anti-pathogenic agents against an existing infection. One option is to create modified plasmids, which upon conjugation (or by another type of transfer) to a harmless host cell, will induce the host cell to produce proteins that will kill other pathogenic bacteria lacking the plasmid. For example, coliform bacteria can carry special plasmids that result in the production of proteins, such as bacteriocins or colicins and microcins, which kill other similar bacterial strains by inducing lysis. The following reference provides further background information concerning these matters and is incorporated by reference—Gillor, O., Kirkup, B. C., Riley, M. A. Colicins and microcins: the next generation antimicrobials. Advanced Applied Microbiology 54, 129-146 (2004). Colicins can kill bacteria by penetrating the cell envelopes, or by increasing the permeability of the cytoplasmic membrane.

Colicins initially bind to receptors on the bacterial surface which also function as receptors for necessary metabolites, therefore it will not be easy for bacteria to eliminate the receptors. For example, the receptors for the colicins B, E, K and M are essential for transporting ferri-enterochelin, vitamin B12, nucleosides, and ferrichrome, respectively. Enterochelin and ferrichrome are iron-chelating agents that enable bacteria to accumulate iron. The following reference provides further background information concerning these matters and is incorporated by reference—Hardy, K. Bacterial Plasmids. 89 (American Society for Microbiology, Washington, D.C., 1986). Colicins A, E1, Ia, Ib and K are bactericidal because they form channels in the cytoplasmic membrane, which become permeable to important ions, such as potassium ions.

Although colicins are produced by E. coli and other members of the Enterobacteriaceae, other Gram-negative and Gram-positive bacterial genera produce analogous proteins that act as bacteriocins. For example, some strains of Streptococcus produce streptococcins, and some strains of Staphylococcus produce staphylococcins. Col plasmids are the plasmids that enable bacteria to produce such bacteriocins and yet be immune to their own antibacterial proteins. The following reference provides further background information concerning these matters and is incorporated by reference—Hardy, K. Bacterial Plasmids. 93 (American Society for Microbiology, Washington, D.C., 1986).

A first strategy would use modified plasmids to essentially act as a genetic Trojan Horse to mutate the recipient pathogen's main chromosomes by deliberately assisting composite transposons to transfer some genes carrying lethal metabolic defect(s) into the pathogen's genotype, such as a defective coding for making bacterial secretion systems such as T3SS or cell wall peptidoglycan synthesis, that could only be lethal to bacteria, not mammals. For example, there are several known genes for bacterial cell wall peptidoglycan synthesis, such as mrbA, murC, murD, murE, murF, ddI, mraY, and murG, and one or more of these genes could be sabotaged by minor modifications. The following reference provides more background information on this matter and is incorporated by reference—Ghuysen, J. M. & Hakenbeck, R. editors. Bacterial Cell Wall. New Comprehensive Biochemistry, 27. New York, N.Y., Elsevier (1994). The defect could either be designed to be latent and affect only the next generation, or designed so that the defect could produce immediate damage that would affect the recipient pathogen itself by shortening its own lifetime and/or at least disrupting the pathogen's reproduction, such as reproduction by the bacterial binary fission process, at one or more steps.

One advantage of using modified plasmids as an agent to attack pathogens, in some of the strategies previously described, is that pathogens that are typically quicker to incorporate new plasmids could be more quickly suppressed by such plasmids. For example, many of the most pathogenic antibiotic resistant bacteria readily accept plasmids, and these bacteria could be more quickly affected by modified, antibacterial plasmids. Furthermore, a great deal is now known about plasmids, and the modification, manufacturing, and purification of plasmids has been extensively discussed in the literature. The following three references provide more background information on these matters and are incorporated by reference—Sambrook, J., & Russell, D. W. Molecular Cloning: A Laboratory Manual. 1.31-1.84 (3rd ed., Cold Spring Harbor Laboratory, Cold Spring Harbor, N.Y., 2001); Neudecker, F. & Grimm, S. High-throughput method for isolating plasmid DNA with reduced lipopolysaccharide content. Biotechniques. 28, 107-109 (2000); and Konecki, D. S. & Phillips, J. J. TurboPrep II: an inexpensive, high-throughput plasmid template preparation protocol. Biotechniques. 24, 286-293 (1998).

A second strategy for using plasmids to attack pathogens would be to create modified plasmids that upon transfer would make the recipient pathogens easier targets for the innate and/or adaptive immune systems of humans to attack. For example, such plasmids could encode for one or more proteins that self-opsonize the recipient pathogens, making it easier for phagocytosis of the pathogens by neutrophils or macrophages. Another option would be creating a plasmid that encodes for protein production that would more quickly or more strongly activate either the innate immune response by creating and secreting specific cytokines for chemotaxis and/or activating phagocytes, such as various combinations of interleukins, or activate the adaptive immune response, either by creating more recognizable antigens, or by creating antigens similar to other common antigens that would activate already existing memory T cells and memory B cells in a human resulting from some previously experienced infection. The mimic antigens for a previously experienced infection could be for a very common infection, or even a recent vaccination specifically intended to create memory T cells and memory B cells to respond to a specific template antigen, the same antigen that would be produced by bacteria carrying the plasmid.

The advantages of utilizing modified plasmids to induce pathogens to trigger the immune system as the actual bacterial killing agent would be numerous and significant. The first advantage is that the host cell or bacteria carrying the modified plasmid itself would not risk immediate self-destruction merely as a result of carrying the modified plasmid during the manufacturing process. There would be no detriment to the host cells or recipient pathogens until the actual immune system attack occurs. Therefore, the modified plasmid itself would be much easier to make in quantity by using host bacterial factories outside of the human body. The second advantage is that the immune system could be stimulated to attack all the pathogens having the same antigens, regardless of whether they actually became recipients of the modified plasmids. The third advantage is that if the adaptive immune system's memory B cells and memory T cells are triggered by bacteria carrying the plasmid, the immune response would typically be at least two to three times faster and stronger than the immune response to a first time infection.

A third strategy would use a large number of modified plasmids to interfere with characteristics that help pathogens in colonization, such as the formation of biofilms. The following reference provides further background information and is incorporated by reference—Ghigo, J. M. Natural conjugative plasmids induce bacterial biofilm development. Nature. 412, 442-445 (2001). Or this strategy could use modified plasmids that interfere with or eliminate the bacterial secretion systems used by pathogenic bacteria to hijack human cells by modifications to the cell membranes and modifications to human immune cells. For example, pathogenic strains of E. coli (such as 0157) and Salmonella use a first type of type 3 secretion system (T3SS) to inject effector proteins into human intestinal cells to help the bacteria adhere or enter the human intestinal cells, and then use a second type of T3SS to hijack immune cells or make them commit suicide. The following three references provide further background information and are incorporated by reference—Finlay, B. B. The art of bacterial warfare. Scientific American. 302, 56-61 (2010); Bhaysar, A. P., Guttman, J. A., Finlay, B. B. Manipulation of host-cell pathways by bacterial pathogens. Nature 449, 827-834 (2007); and Finlay, B. B. & McFadden, G. Anti-immunology: evasion of the host immune system by bacterial and viral pathogens. Cell 124, 767-782 (2006).

For example, a second type of T3SS used by Salmonella typhi can convert immune cell phagocytic vacuoles into bacterial incubators for Salmonella typhi to cause typhoid fever. Yersinia pestis, which causes bubonic plague, also uses a type of T3SS to inject effector proteins into immune cell macrophages to deactivate the macrophages. Shigella bacteria, such as Shigella dysenteriae, even use T3SS to penetrate human cells and avoid contact with immune cells and entirely avoid antibody creation by preventing phagocytic cells from presenting antigens to lymphocytes. The following three references provide more background information on this matter and are incorporated by reference—Finlay, B. B. & McFadden, G. Anti-immunology: evasion of the host immune system by bacterial and viral pathogens. Cell 124, 767-782 (2006); Croxen, M. A., Finlay, B. B. Molecular mechanisms of Escherichia coli pathogenicity. Nature Reviews Microbiology. 8, 26-38 (2010); and Rosenberger, C. M. & Finlay, B. B. Phagocyte sabotage: disruption of macrophage signaling by bacterial pathogens. Nature Reviews Molecular Cell Biology 4, 385-396 (2003).

Specifically targeted anti-secretion system plasmids would be harmless to humans. The modified plasmid coding would also need to be carefully selected to avoid giving any true benefit to pathogenic bacteria. Such anti-secretion system plasmids should be easily transferred to pathogens by conjugation or transformation.

Besides the first strategy of using modified plasmids to sabotage the genome of a pathogen by using modified plasmids and composite transposons to sabotage the genome of a pathogen, the second strategy of using modified plasmids to make pathogens easier targets for the innate and adaptive immune systems of humans, and the third strategy of using modified plasmids to disrupt pathogen colonization, in addition there are two other strategies for using modified plasmids to fight pathogens.

A fourth strategy would involve injecting a large quantity of host cells with modified plasmids in the vicinity of pathogens, for conjugation or other transfer of the plasmids to the pathogens. These modified plasmids could encode the production of some protein that would disrupt some vital metabolic function of the pathogen, or alternatively encode the production of proteins that would create an overwhelming metabolic load in energy or materials for the recipient pathogen, thus reducing their capability to take up or metabolically support other plasmids that actually encode antibiotic resistance. For example, a bacterium cannot metabolically support an excessive number of plasmids under any circumstances, and modified plasmids that disrupt metabolic functions, or simply create an overwhelming metabolic load in their use of energy or materials, would naturally target any bacterium that is most receptive to plasmids, whether the bacterium is harmless or pathogenic. The following reference provides further background information and is incorporated by reference—Hayes, F. E. coli Plasmid Vectors Methods and Applications. (ed. Casali, N., & Preston, A.) 1-18 (Humana Press, Totowa, N.J., 2003). One advantage of this strategy is that such modified plasmids would not need to be designed for preferential uptake by pathogenic bacteria compared to harmless bacteria. Any pathogens that take up such modified plasmids would be killed, or at least suppressed. One challenge would be the manufacturing of the modified plasmid, unless it could be produced in an inactive form in a host cell factory and then later activated.

The fifth strategy utilizes the creation of modified plasmids that can be easily taken up by relatively harmless bacteria or other donor cells, which would produce bacteriocin-like proteins to kill bacteria that are pathogenic to humans. The ideal antibacterial plasmid would be easy to produce in quantity, stable enough for easy transport and use, easily taken up by relatively harmless bacteria, selective enough not to be taken up by pathogenic bacteria, and extremely lethal to pathogenic bacteria, yet harmless to humans. Since bacteria have cell walls and mammals do not, a relatively safe plasmid would code for at least one protein to cause a major disruption to bacterial cell walls, affecting either the synthesis of new bacterial cell walls, or perforating existing bacterial cell walls. Such proteins are could actually be designed to mimic known bacteriocins, or alternatively, they could mimic the actions of proteins used by the complement system of the human innate immune system, or mimic the perform and granzyme proteins of the NK cells of the human innate immune system and by cytotoxic T cells of the human adaptive immune system.

The largest challenge with this particular strategy would be custom-tailoring a plasmid that would be preferentially taken up by harmless bacteria, and yet not be acquired by pathogens. It may be more feasible to design customized host cells, such as bacteria, that carry the plasmids that code for lethal protein production that would not transfer the plasmids to pathogens to any significant extent, but merely use only the customized host cells themselves as factories to produce the lethal proteins that attack pathogens.

### Experiment

An experiment to investigate the conjugative transfer of an RP4 plasmid conferring resistance to tetracycline from one E. coli donor strain to another tetracycline vulnerable E. coli host strain at human body temperature was conducted. The RP4 plasmid was an attractive plasmid choice due to its robust persistence in E. coli and very broad conjugative transfer characteristics to many different micro-organisms. A base pair count and a restriction map for the RP4 plasmid can be found in the following background reference which is incorporated by reference—Bukhari, A. I., Shapiro, J. A., Adhya, S. L. DNA Insertion Elements, Plasmids, and Episomes. 678-679 (1st ed., Cold Spring Harbor Laboratory, Cold Spring Harbor, N.Y., 1977).

Petri dishes containing glucose-supplemented nutrient agar for E. coli were made. A fixed number of tetracycline vulnerable E. coli colonies were streaked on the Petri dishes and briefly incubated at 25 degrees C. Then a second RP4 plasmid solution of E. coli was streaked perpendicularly to the first host colonies of E. coli. Both the tetracycline vulnerable E. coli colonies and RP4 plasmid E. coli colonies were resistant to kanamycin. Then the Petri dishes were incubated at 25 degrees C. for one hour for conjugative transfer and plated onto two different Petri dishes, using conventional replica plating techniques. Further background information on replica plating is explained in the following reference which is incorporated by reference—Lederberg, J. & Lederberg, E. M. Replica Plating and Indirect Selection of Bacterial Mutants. Journal of Bacteriology. 63, 399-406 (1952). The replica plating transfer was achieved by plating a previously sterilized disk of filter paper with a diameter slightly less than the 100 millimeter diameter of the original Petri dish where the conjugation occurred. The first replica plating Petri dish had a tetracycline infused media (50 micrograms of tetracycline per milliliter of standard growth media). The second replica plating Petri dish had a kanamycin infused media (50 micrograms of kanamycin per milliliter of standard growth media). Then both types of Petri dishes were incubated at human body temperature 37 C for 8 hours. The number of colonies on the tetracycline infused Petri dishes and the number of colonies on the kanamycin infused Petri dishes were compared. The percentage of colonies that represent successful RP4 plasmid conjugative transfer from donor cell to host cell could be determined by comparing the colonies on the tetracycline infused Petri dishes to the colonies on the kanamycin infused Petri dishes.

A high rate of conjugative transfer of the RP4 plasmid from donor E. coli to host E. coli demonstrated in principle the feasibility of using a modified RP4 plasmid as a plasmid vehicle for antibacterial or other anti-pathogen applications utilizing the previously discussed strategies.

In the following flowcharts of methods to use modified plasmids in donor micro-organisms for a beneficial use, the word “transfer” could be defined to include conjugation, transduction or transfection. In the discussion below, the phrase “facilitating the transfer of the least one type of modified plasmid” can mean merely waiting for the transfer to happen over time, or actively helping the transfer to occur by the introduction of other agents or by otherwise changing the environment for the donor or host micro-organism (e.g., changing the temperature, sugar levels, or some other equivalent parameter that would promote the transfer). The word “micro-organism” could be defined to include bacteria, fungi, protozoa, parasites, or other micro-organisms. The word “organism” could be defined to include any type of animal such as a mammal, or even could include any type of plant, such plants used for agriculture.

In the following flowcharts of methods to use modified plasmids in donor micro-organisms for a beneficial use, the at least one type of host micro-organism in the organism may or may not be identical to the one type of species of micro-organism that is pathological. In one embodiment, the host micro-organism is also the micro-organism that is pathological. In another embodiment, the host micro-organism is different from the micro-organism that is pathological. In the discussion below, “beneficial use” may be a medical use, or in another embodiment it may be to help other organisms fight or attack a pathogen causing trouble in agriculture. In the discussion below, “suppress” may mean killing a pathogen in one embodiment, in another embodiment it may mean to weaken or slow the growth rate of a pathogen, in another embodiment it may mean to reduce the population of the pathogen, or in another embodiment it may mean to make the pathogen more easily attacked by another beneficial agent.

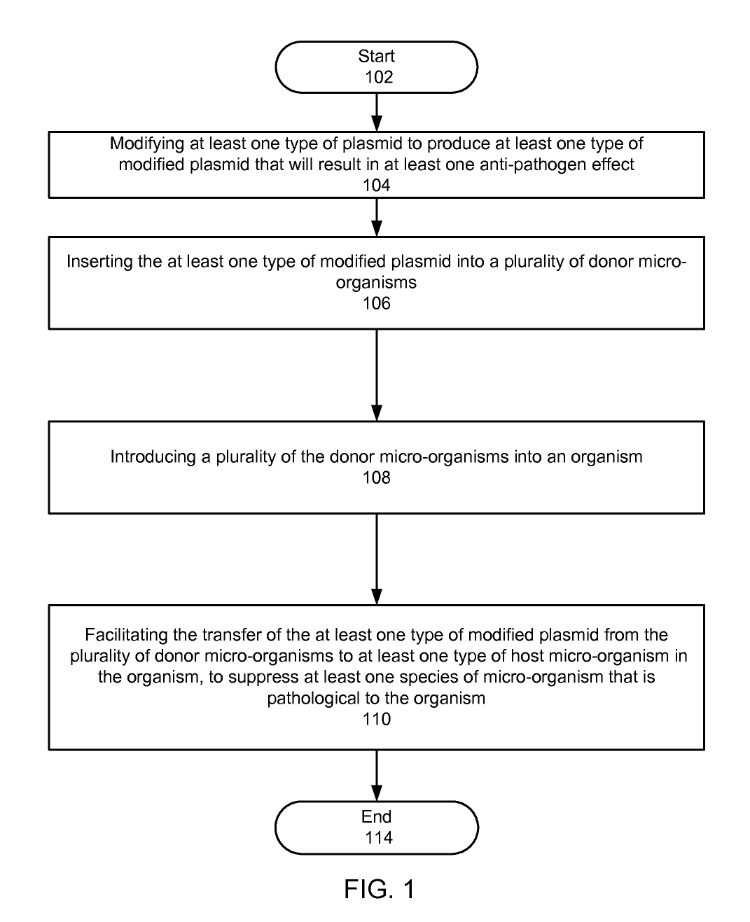

FIG. 1 illustrates a flowchart of a method to use modified plasmids in donor micro-organisms for a beneficial use, in accordance with one embodiment of the invention. As previously discussed above, potential candidate plasmids suitable for modification and use against specific pathogens can be pre-identified from a vast scientific literature and then commercially purchased inside some carrier micro-organism from many microbiology supply sources, or in many cases alternatively acquired at little or no cost from university microbiology department researchers that recently studied the same plasmids of interest. The techniques for modification of plasmids are also very well known and have been described in detail in a vast scientific literature. The method starts in operation 102. Operation 104 is next and includes modifying at least one type of plasmid to produce at least one type of modified plasmid that will result in at least one anti-pathogen effect. Operation 106 is next and includes inserting the at least one type of modified plasmid into a plurality of donor micro-organisms. Operation 108 is next and includes introducing a plurality of the donor micro-organisms into an organism. Operation 110 is next and includes facilitating the transfer of the at least one type of modified plasmid from the plurality of donor micro-organisms to at least one type of host micro-organism in the organism, to suppress at least one type of species of micro-organism that is pathological to the organism. The method ends in operation 114.

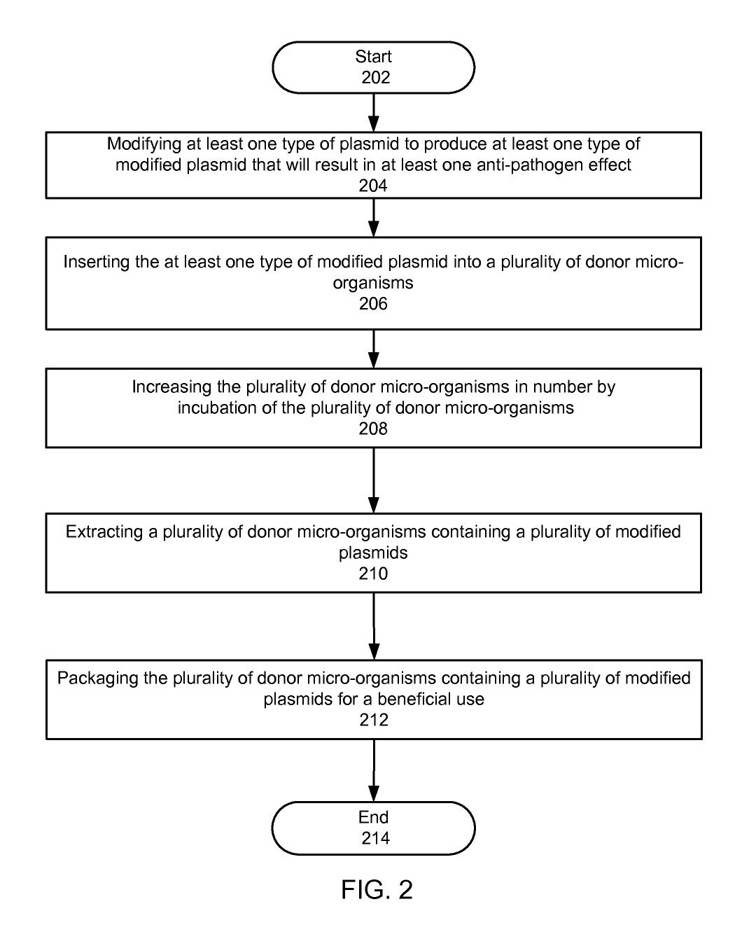

FIG. 2 illustrates a flowchart of a method to manufacture modified plasmids in donor micro-organisms for a beneficial use, in accordance with one embodiment of the invention. The method starts in operation 202. Operation 204 is next and includes modifying at least one type of plasmid to produce at least one type of modified plasmid that will result in at least one anti-pathogen effect. Operation 206 is next and includes inserting the at least one type of modified plasmid into a plurality of donor micro-organisms. Operation 208 is next and includes increasing the plurality of donor micro-organisms in number by incubation of the plurality of donor micro-organisms. The incubation may optionally include incubation in a growth medium favorable to the donor micro-organisms. Operation 210 is next and includes extracting a plurality of donor micro-organisms containing a plurality of modified plasmids. Operation 212 is next and includes packaging the plurality of donor micro-organisms containing the plurality of modified plasmids for a beneficial use. The method ends in operation 214.

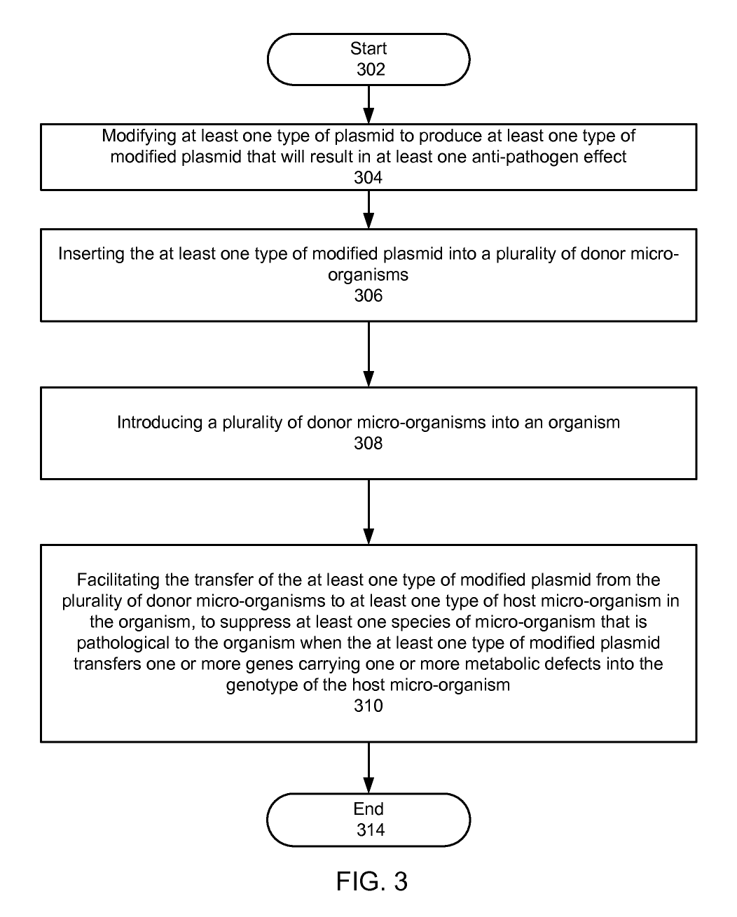

FIG. 3 illustrates a flowchart of a method to use modified plasmids in donor micro-organisms for a beneficial use, in accordance with another embodiment of the invention. The method starts in operation 302. Operation 304 is next and includes modifying at least one type of plasmid to produce at least one type of modified plasmid that will result in at least one anti-pathogen effect. Operation 306 is next and includes inserting the at least one type of modified plasmid into a plurality of donor micro-organisms. Operation 308 is next and includes introducing a plurality of the donor micro-organisms into an organism. Operation 310 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes carrying one or more metabolic defects into the genotype of the host micro-organism. The method ends in operation 314.

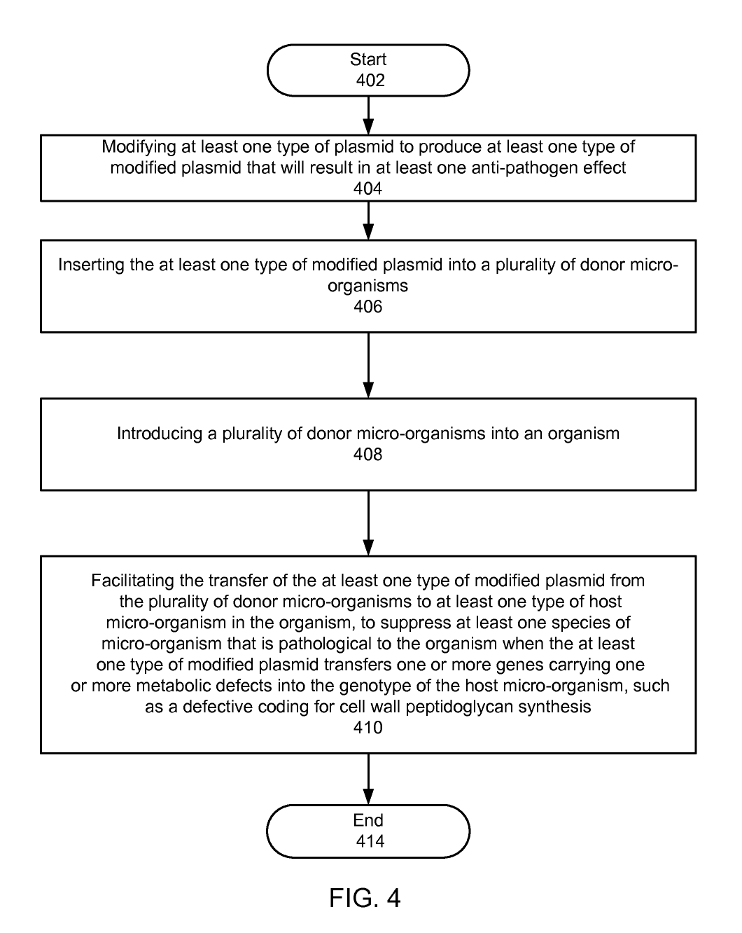

FIG. 4 illustrates a flowchart of a method to use modified plasmids in donor micro-organisms for a beneficial use, in accordance with another embodiment of the invention. The method starts in operation 402. Operation 404 is next and includes modifying at least one type of plasmid to produce at least one type of modified plasmid that will result in at least one anti-pathogen effect. Operation 406 is next and includes inserting the at least one type of modified plasmid into a plurality of donor micro-organisms. Operation 408 is next and includes introducing a plurality of the donor micro-organisms into an organism. Operation 410 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes carrying one or more metabolic defects into the genotype of the host micro-organism, such as a defective coding for cell wall peptidoglycan synthesis. The method ends in operation 414.

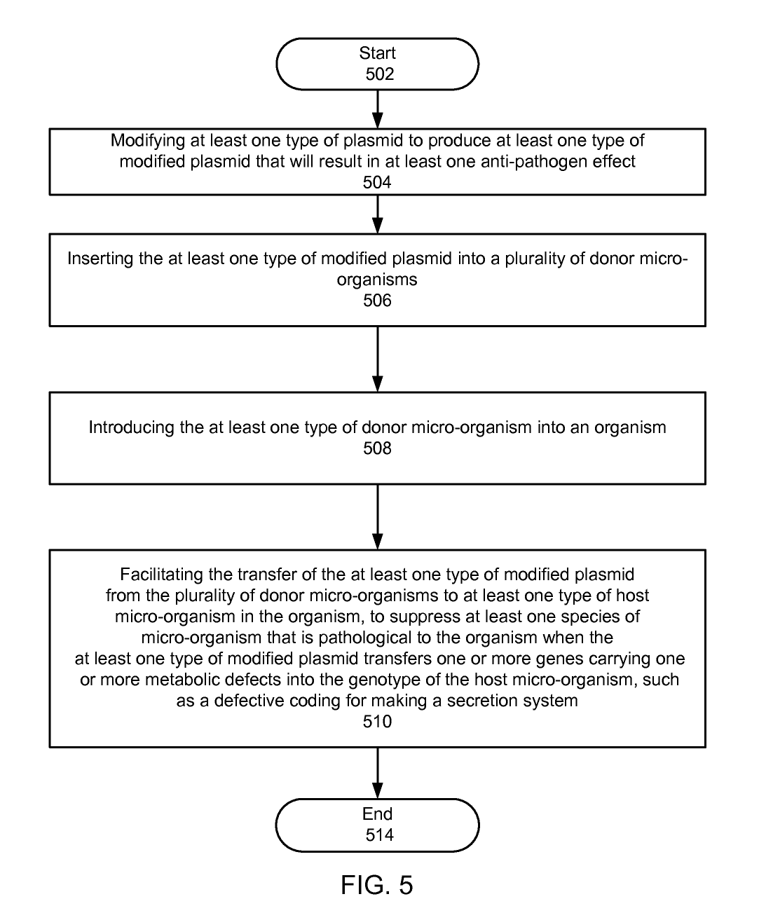

FIG. 5 illustrates a flowchart of a method to use modified plasmids in donor micro-organisms for a beneficial use, in accordance with another embodiment of the invention. The method starts in operation 502. Operation 504 is next and includes modifying at least one type of plasmid to produce at least one type of modified plasmid that will result in at least one anti-pathogen effect. Operation 506 is next and includes inserting the at least one type of modified plasmid into a plurality of donor micro-organisms. Operation 508 is next and includes introducing a plurality of the donor micro-organisms into an organism. Operation 510 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes carrying one or more metabolic defects into the genotype of the host micro-organism, such as a defective coding for making a secretion system (e.g., a bacterial secretion system, or equivalent). The method ends in operation 514.

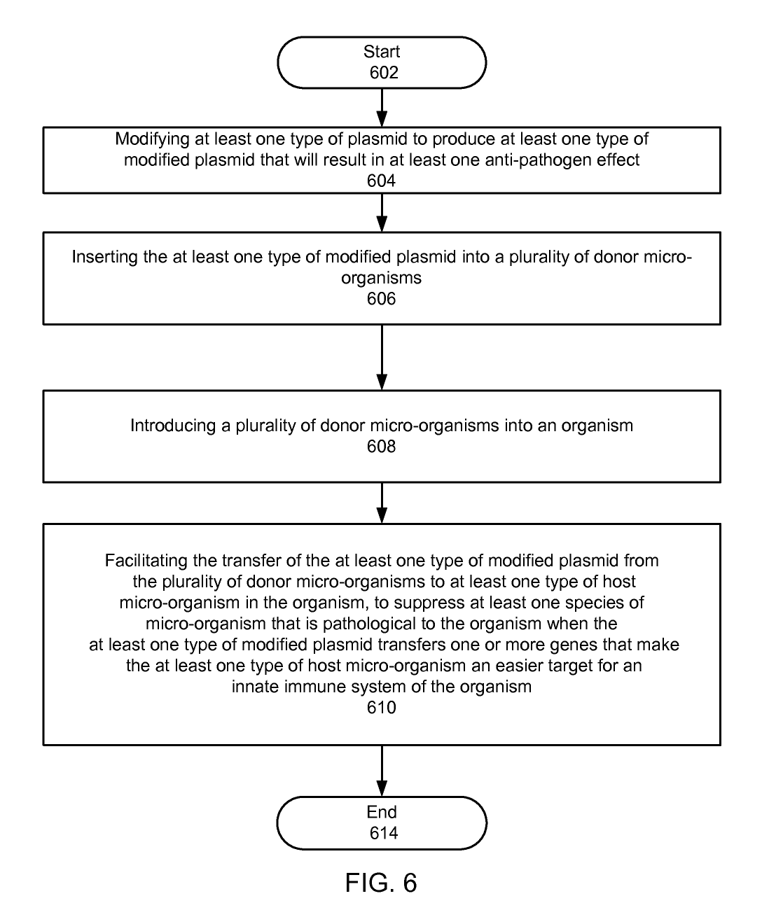

FIG. 6 illustrates a flowchart of a method to use modified plasmids in donor micro-organisms for a beneficial use, in accordance with another embodiment of the invention. The method starts in operation 602. Operation 604 is next and includes modifying at least one type of plasmid to produce at least one type of modified plasmid that will result in at least one anti-pathogen effect. Operation 606 is next and includes inserting the at least one type of modified plasmid into a plurality of donor micro-organisms. Operation 608 is next and includes introducing a plurality of the donor micro-organisms into an organism. Operation 610 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes that make the at least one type of host micro-organism an easier target for an innate immune system of the organism. The method ends in operation 614.

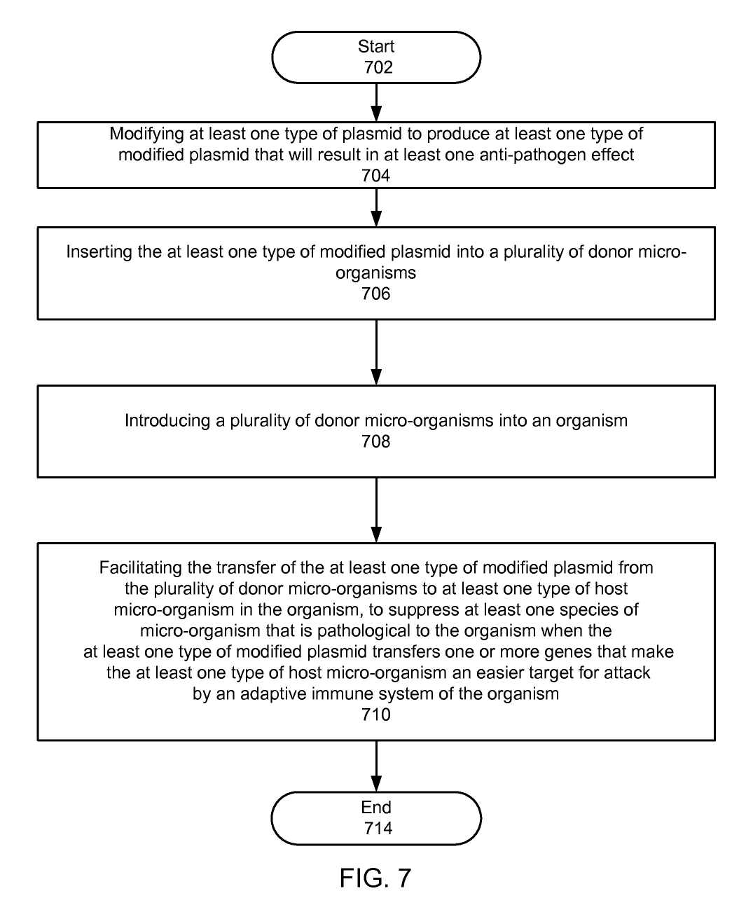

FIG. 7 illustrates a flowchart of a method to use modified plasmids in donor micro-organisms for a beneficial use, in accordance with another embodiment of the invention. The method starts in operation 702. Operation 704 is next and includes modifying at least one type of plasmid to produce at least one type of modified plasmid that will result in at least one anti-pathogen effect. Operation 606 is next and includes inserting the at least one type of modified plasmid into a plurality of donor micro-organisms. Operation 708 is next and includes introducing a plurality of the donor micro-organisms into an organism. Operation 710 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes that make the at least one type of host micro-organism an easier target for an adaptive immune system of the organism. The method ends in operation 714.

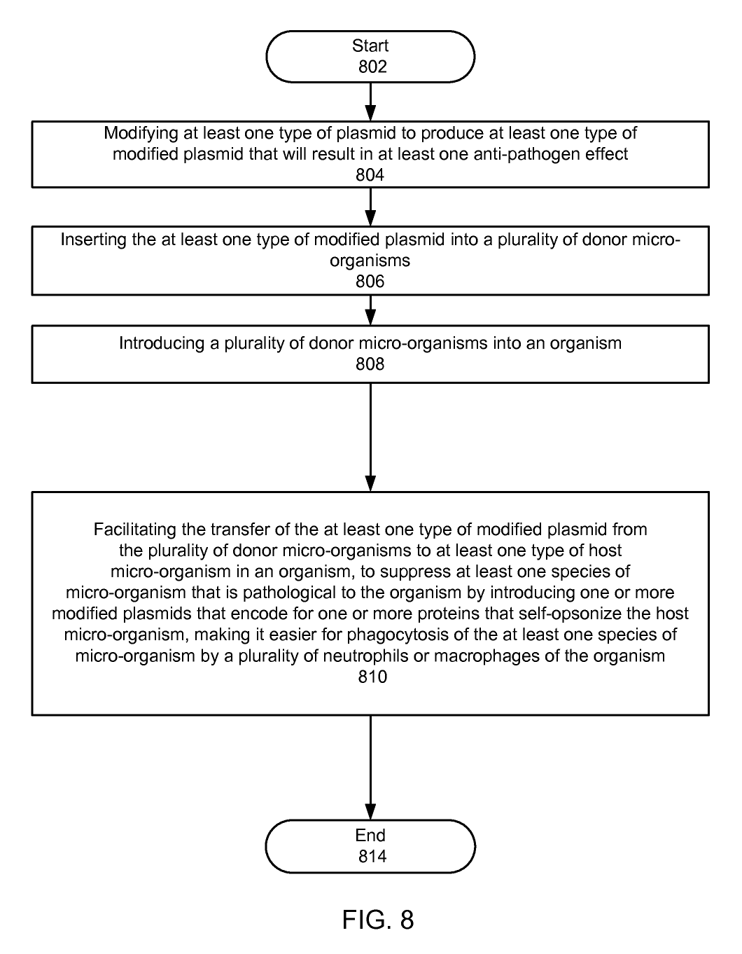

FIG. 8 illustrates a flowchart of a method to use modified plasmids in donor micro-organisms for a beneficial use, in accordance with another embodiment of the invention. The method starts in operation 802. Operation 804 is next and includes modifying at least one type of plasmid to produce at least one type of modified plasmid that will result in at least one anti-pathogen effect. Operation 806 is next and includes inserting the at least one type of modified plasmid into a plurality of donor micro-organisms. Operation 808 is next and includes introducing a plurality of the donor micro-organisms into an organism. Operation 810 is next and includes facilitating the transfer of the at least one type of modified plasmid from the plurality of donor micro-organisms to at least one type of host micro-organism in the organism, to suppress at least one type of species of micro-organism that is pathological to the organism by introducing one or more modified plasmids that encode for one or more proteins that self-opsonize the host micro-organism, making it easier for phagocytosis of the at least one species of micro-organism by a plurality of neutrophils or macrophages of the organism. The method ends in operation 814.

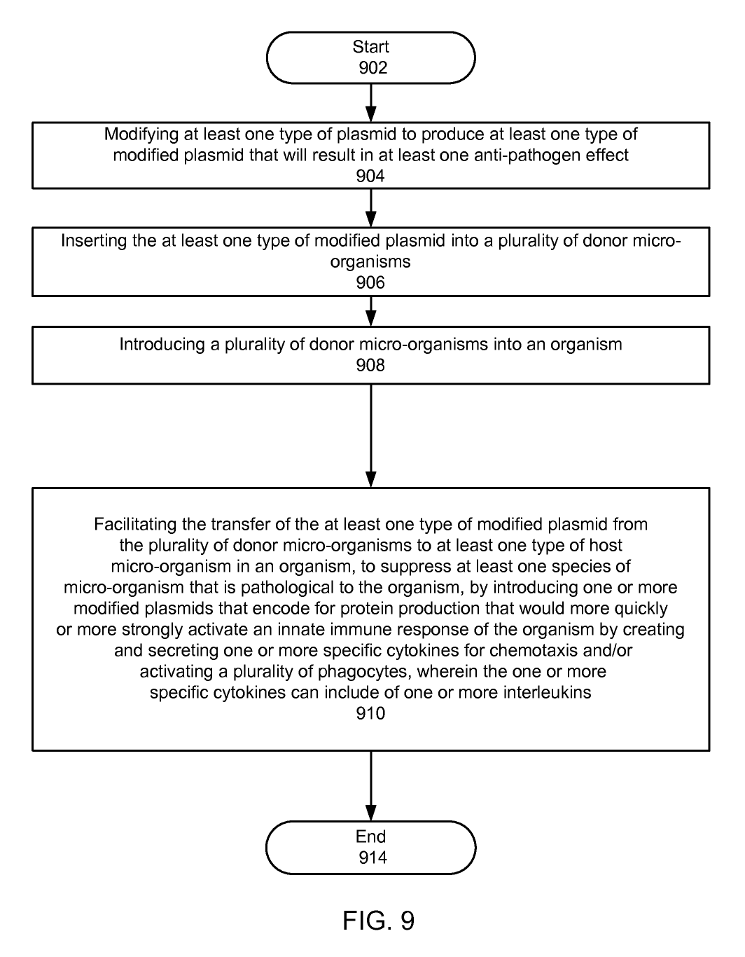

FIG. 9 illustrates a flowchart of a method to use modified plasmids in donor micro-organisms for a beneficial use, in accordance with another embodiment of the invention. The method starts in operation 902. Operation 904 is next and includes modifying at least one type of plasmid to produce at least one type of modified plasmid that will result in at least one anti-pathogen effect. Operation 906 is next and includes inserting the at least one type of modified plasmid into a plurality of donor micro-organisms. Operation 908 is next and includes introducing a plurality of the donor micro-organisms into an organism. Operation 910 is next and includes facilitating the transfer of the at least one type of modified plasmid from the plurality of donor micro-organisms to at least one type of host micro-organism in the organism, to suppress at least one type of species of micro-organism that is pathological to the organism, by introducing one or more plasmids that encode for protein production that would more quickly or more strongly activate an innate immune system of the organism by creating and secreting one or more specific cytokines for chemotaxis and/or activating a plurality of phagocytes, wherein the one or more specific cytokines can include one or more interleukins. The method ends in operation 914.

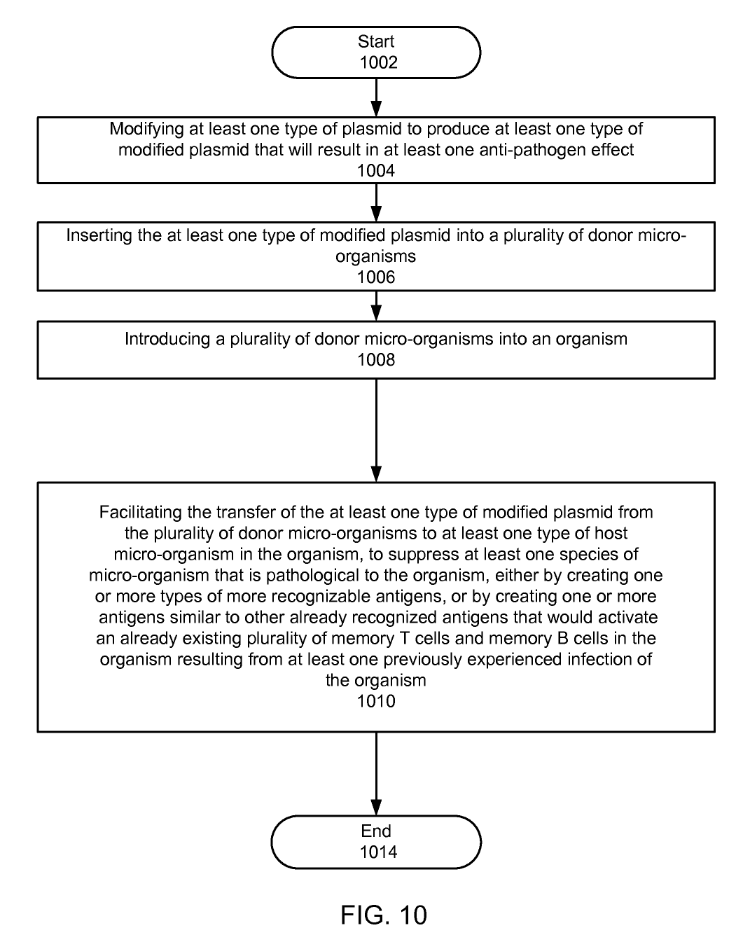

FIG. 10 illustrates a flowchart of a method to use modified plasmids in donor micro-organisms for a beneficial use, in accordance with another embodiment of the invention. The method starts in operation 1002. Operation 1004 is next and includes modifying at least one type of plasmid to produce at least one type of modified plasmid that will result in at least one anti-pathogen effect. Operation 1006 is next and includes inserting the at least one type of modified plasmid into a plurality of donor micro-organisms. Operation 1008 is next and includes introducing a plurality of the donor micro-organisms into an organism. Operation 1010 is next and includes facilitating the transfer of the at least one type of modified plasmid from the plurality of donor micro-organisms to at least one type of host micro-organism in the organism, to suppress at least one type of species of micro-organism that is pathological to the organism, either by creating one or more types of more recognizable antigens, or by creating one or more antigens similar to other already recognized antigens that would activate an already existing plurality of memory T cells and memory B cells in the organism resulting from at least one previously experienced infection of the organism. The method ends in operation 1014.

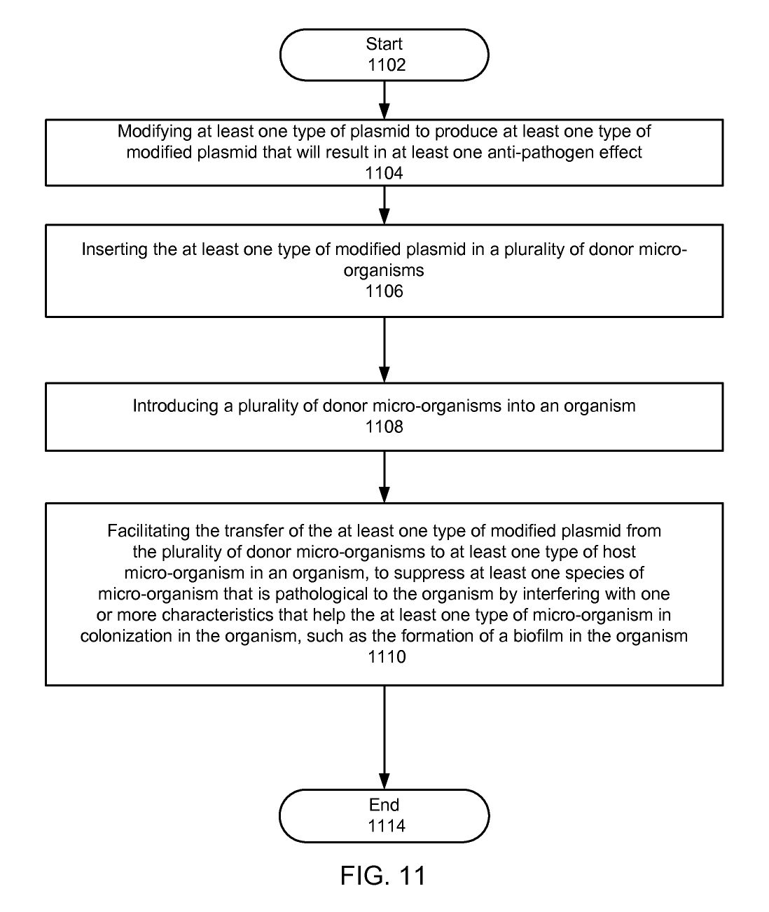

FIG. 11 illustrates a flowchart of a method to use modified plasmids in donor micro-organisms for a beneficial use, in accordance with another embodiment of the invention. The method starts in operation 1102. Operation 1104 is next and includes modifying at least one type of plasmid to produce at least one type of modified plasmid that will result in at least one anti-pathogen effect. Operation 1106 is next and includes inserting the at least one type of modified plasmid into a plurality of donor micro-organisms. Operation 1108 is next and includes introducing a plurality of the donor micro-organisms into an organism. Operation 1110 is next and includes facilitating the transfer of the at least one type of modified plasmid from the plurality of donor micro-organisms to at least one type of host micro-organism in the organism, to suppress at least one type of species of micro-organism that is pathological to the organism by interfering with one or more characteristics that help the at least one type of micro-organism in colonization in the organism, such the formation of a biofilm in the organism. The method ends in operation 1114.

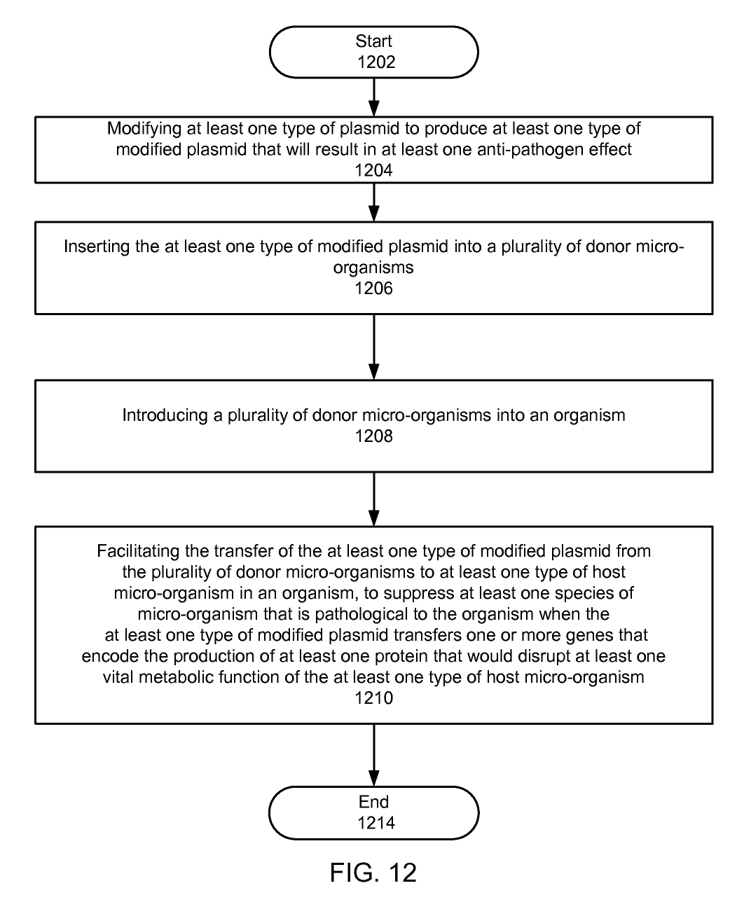

FIG. 12 illustrates a flowchart of a method to use modified plasmids in donor micro-organisms for a beneficial use, in accordance with another embodiment of the invention. The method starts in operation 1202. Operation 1204 is next and includes modifying at least one type of plasmid to produce at least one type of modified plasmid that will result in at least one anti-pathogen effect. Operation 1206 is next and includes inserting the at least one type of modified plasmid into a plurality of donor micro-organisms. Operation 1208 is next and includes introducing a plurality of the donor micro-organisms into an organism. Operation 1210 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes that encode the production of at least one protein that would disrupt at least one vital metabolic function of the at least one type of host micro micro-organism. The method ends in operation 1214.

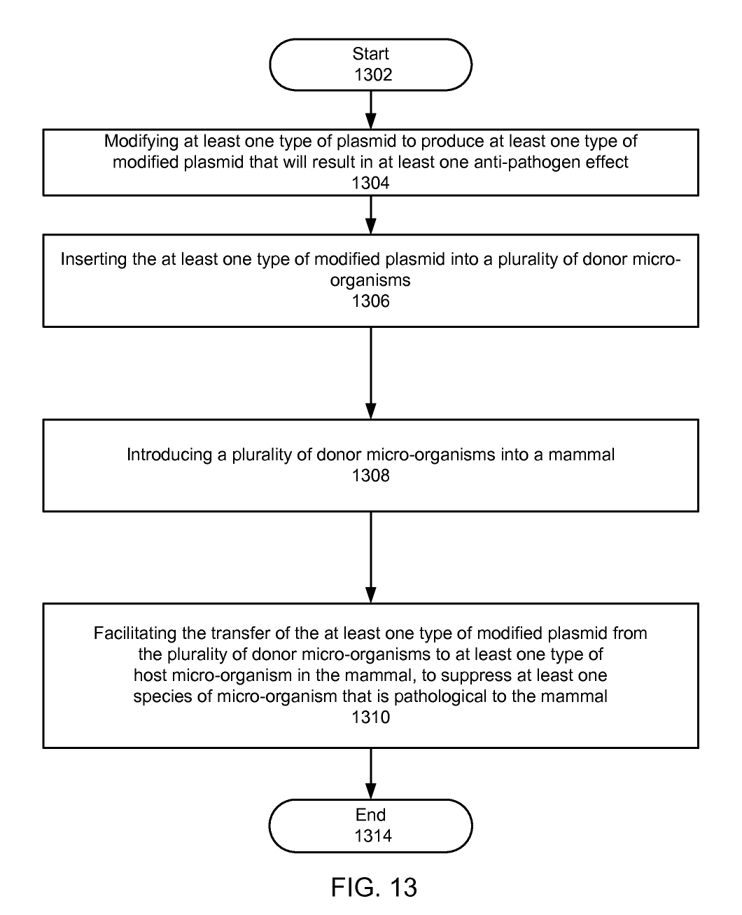

FIG. 13 illustrates a flowchart of a method to use modified plasmids in donor micro-organisms for a beneficial use, in accordance with another embodiment of the invention. The method starts in operation 1302. Operation 1304 is next and includes modifying at least one type of plasmid to produce at least one type of modified plasmid that will result in at least one anti-pathogen effect. Operation 1306 is next and includes inserting the at least one type of modified plasmid into a plurality of donor micro-organisms. Operation 1308 is next and includes introducing a plurality of the donor micro-organisms into a mammal. Operation 610 is next and includes facilitating the transfer of the at least one type of modified plasmid from the plurality of donor micro-organisms to at least one type of host micro-organism in the mammal, to suppress at least one type of species of micro-organism that is pathological to the mammal. The method ends in operation 1314.

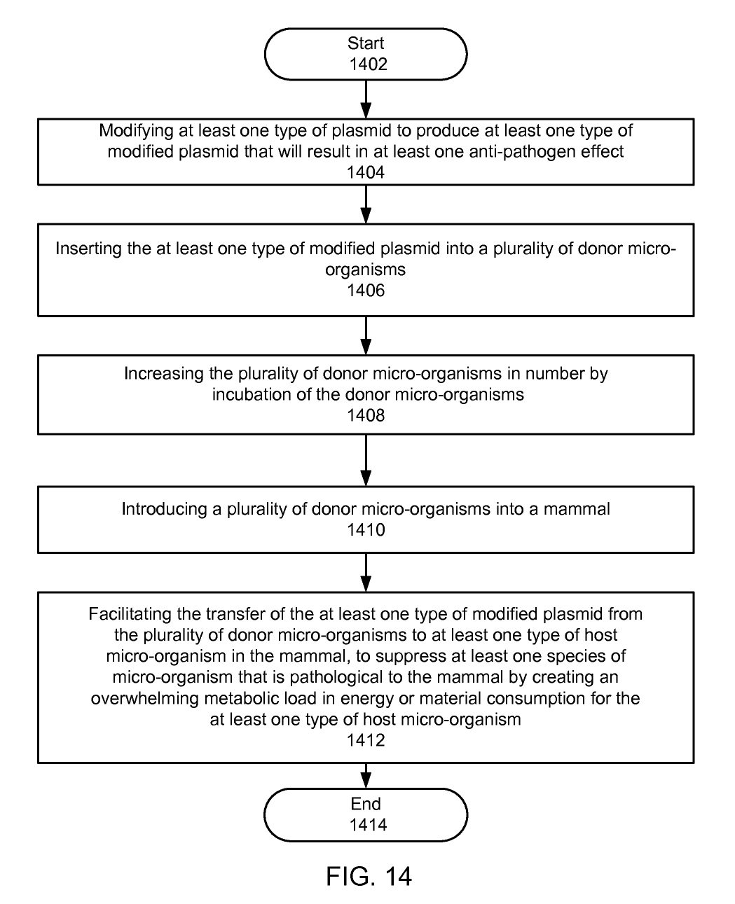

FIG. 14 illustrates a flowchart of a method to use modified plasmids in donor micro-organisms for a beneficial use, in accordance with another embodiment of the invention. The method starts in operation 1402. Operation 1404 is next and includes modifying at least one type of plasmid to produce at least one type of modified plasmid that will result in at least one anti-pathogen effect. Operation 1406 is next and includes inserting the at least one type of modified plasmid into a plurality of donor micro-organisms. Operation 1408 is next and includes increasing the plurality of donor micro-organisms in number by incubation of the donor micro-organisms. Operation 1410 is next and includes introducing a plurality of the donor micro-organisms into a mammal. Operation 1412 is next and includes facilitating the transfer of the at least one type of modified plasmid from the plurality of donor micro-organisms to at least one type of host micro-organism in the mammal, to suppress at least one type of species of micro-organism that is pathological to the mammal by creating an overwhelming metabolic load in energy or material consumption for the at least one type of host micro-organism. The method ends in operation 1414.

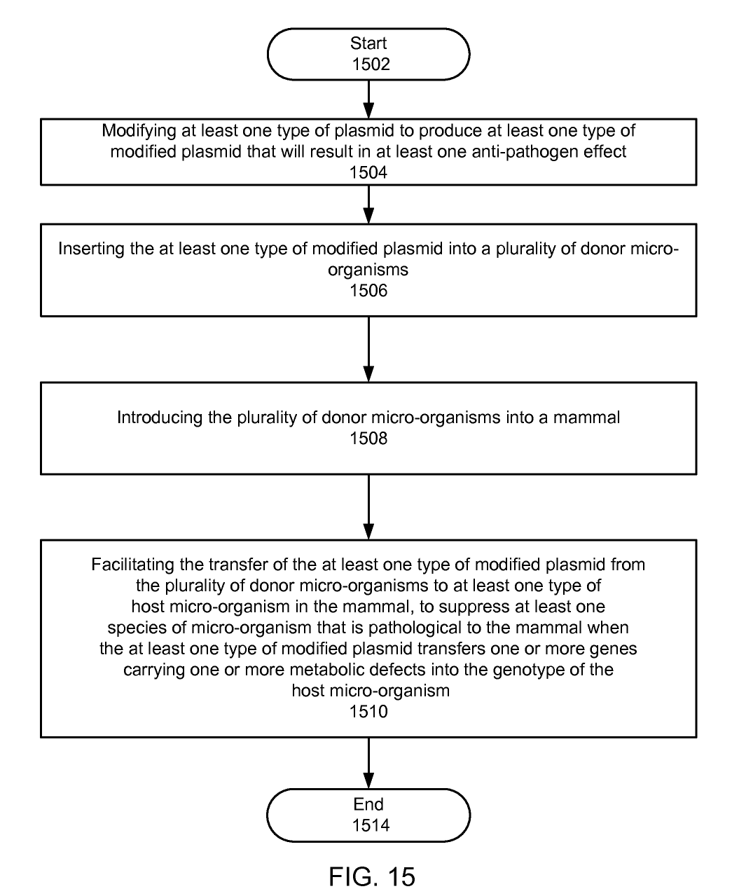

FIG. 15 illustrates a flowchart of a method to use modified plasmids in donor micro-organisms for a beneficial use, in accordance with another embodiment of the invention. The method starts in operation 1502. Operation 1504 is next and includes modifying at least one type of plasmid to produce at least one type of modified plasmid that will result in at least one anti-pathogen effect. Operation 1506 is next and includes inserting the at least one type of modified plasmid into a plurality of donor micro-organisms. Operation 1508 is next and includes introducing a plurality of the donor micro-organisms into a mammal. Operation 1510 is next and includes facilitating the transfer of the at least one type of modified plasmid from the plurality of donor micro-organisms to at least one type of host micro-organism in the mammal, to suppress at least one type of species of micro-organism that is pathological to the mammal when the at least one type of modified plasmid transfers one or more genes carrying one or more metabolic defects into the genotype of the host micro-organism. The method ends in operation 1514.

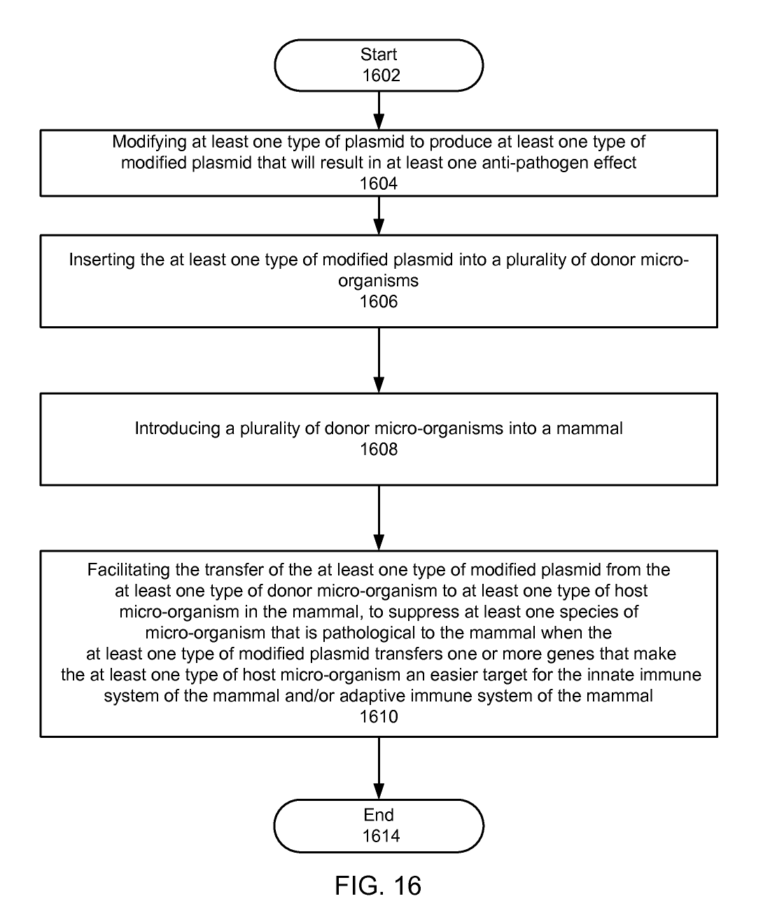

FIG. 16 illustrates a flowchart of a method to use modified plasmids in donor micro-organisms for a beneficial use, in accordance with another embodiment of the invention. The method starts in operation 1602. Operation 1604 is next and includes modifying at least one type of plasmid to produce at least one type of modified plasmid that will result in at least one anti-pathogen effect. Operation 1606 is next and includes inserting the at least one type of modified plasmid into a plurality of donor micro-organisms. Operation 1608 is next and includes introducing a plurality of the donor micro-organisms into an organism. Operation 1610 is next and includes facilitating the transfer of the at least one type of modified plasmid from the plurality of donor micro-organisms to at least one type of host micro-organism in the organism, to suppress at least one type of species of micro-organism that is pathological to the organism when the at least one type of modified plasmid transfers one or more genes that make the at least one type of host micro-organism an easier target for an innate immune system of the mammal and/or an adaptive immune system of the mammal. The method ends in operation 1614.

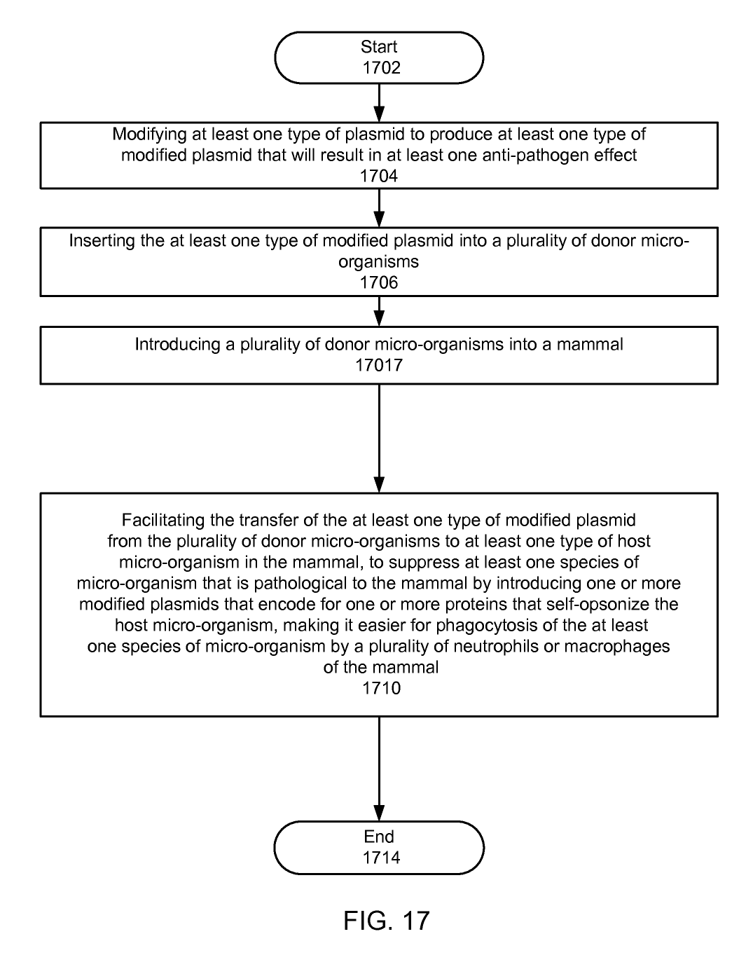

FIG. 17 illustrates a flowchart of a method to use modified plasmids in donor micro-organisms for a beneficial use, in accordance with another embodiment of the invention. The method starts in operation 1702. Operation 1704 is next and includes modifying at least one type of plasmid to produce at least one type of modified plasmid that will result in at least one anti-pathogen effect. Operation 1706 is next and includes inserting the at least one type of modified plasmid into a plurality of donor micro-organisms. Operation 1708 is next and includes introducing a plurality of the donor micro-organisms into a mammal. Operation 1710 is next and includes facilitating the transfer of the at least one type of modified plasmid from the plurality of donor micro-organisms to at least one type of host micro-organism in the mammal, to suppress at least one type of species of micro-organism that is pathological to the mammal by introducing one or more modified plasmids that encode for one or more proteins that self-opsonize the host micro-organism, making it easier for phagocytosis of the least one species of micro-organism by a plurality of neutrophils or macrophages of the mammal. The method ends in operation 1714.

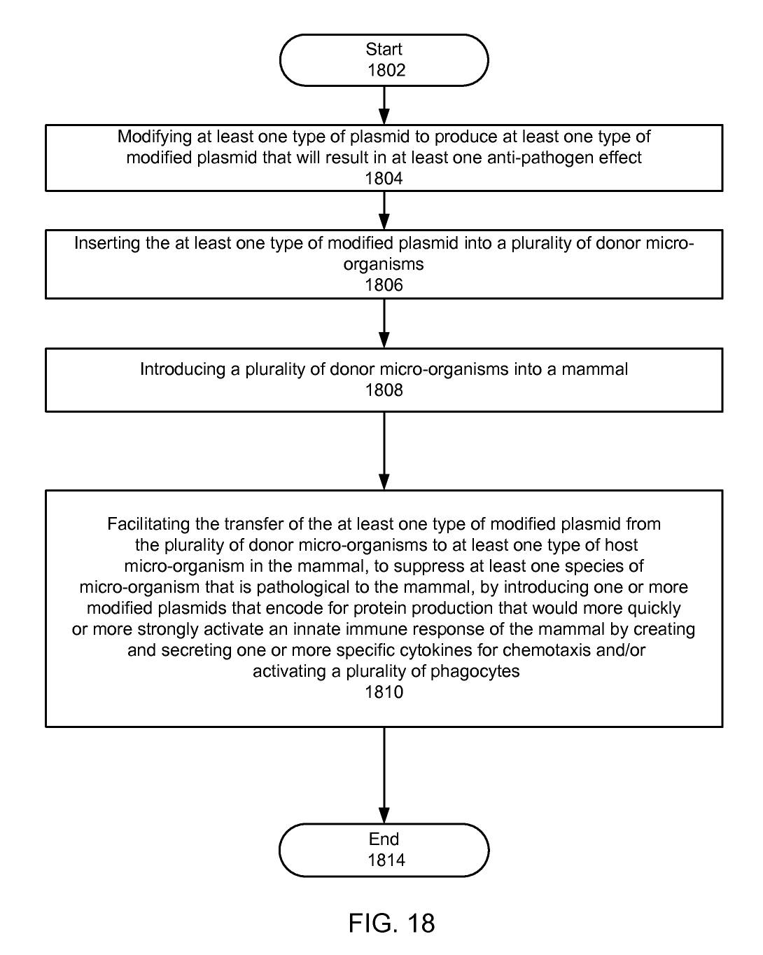

FIG. 18 illustrates a flowchart of a method to use modified plasmids in donor micro-organisms for a beneficial use, in accordance with another embodiment of the invention. The method starts in operation 1802. Operation 1804 is next and includes modifying at least one type of plasmid to produce at least one type of modified plasmid that will result in at least one anti-pathogen effect. Operation 1806 is next and includes inserting the at least one type of modified plasmid into a plurality of donor micro-organisms. Operation 1808 is next and includes introducing a plurality of the donor micro-organisms into a mammal. Operation 1810 is next and includes facilitating the transfer of the at least one type of modified plasmid from the plurality of donor micro-organisms to at least one type of host micro-organism in the mammal, to suppress at least one type of species of micro-organism that is pathological to the mammal, by introducing one or more modified plasmids that encode for protein production that would more quickly or more strongly activate an innate immune system of the mammal by creating and secreting one or more specific cytokines for chemotaxis and/or activating a plurality of phagocytes. In one embodiment of the invention, the one or more specific cytokines can include one or more interleukins. The method ends in operation 1814.

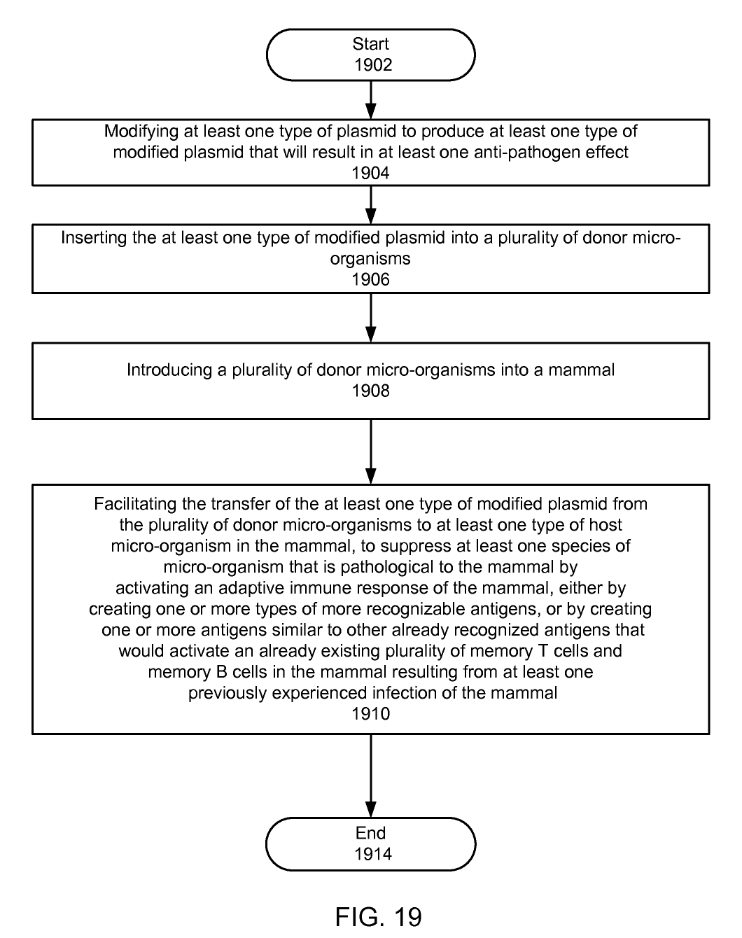

FIG. 19 illustrates a flowchart of a method to use modified plasmids in donor micro-organisms for a beneficial use, in accordance with another embodiment of the invention. The method starts in operation 1902. Operation 1904 is next and includes modifying at least one type of plasmid to produce at least one type of modified plasmid that will result in at least one anti-pathogen effect. Operation 1906 is next and includes inserting the at least one type of modified plasmid into a plurality of donor micro-organisms. Operation 1908 is next and includes introducing a plurality of the donor micro-organisms into a mammal. Operation 1910 is next and includes facilitating the transfer of the at least one type of modified plasmid from the plurality of donor micro-organisms to at least one type of host micro-organism in the mammal, to suppress at least one type of species of micro-organism that is pathological to the mammal by activating an adaptive immune system of the mammal, either by creating one or more types of more recognizable antigens, or by creating one or more antigens similar to other already recognized antigens that would activate an already existing plurality of memory T cells and memory B cells in the mammal resulting from at least one previously experience infection of the mammal. The method ends in operation 1914.

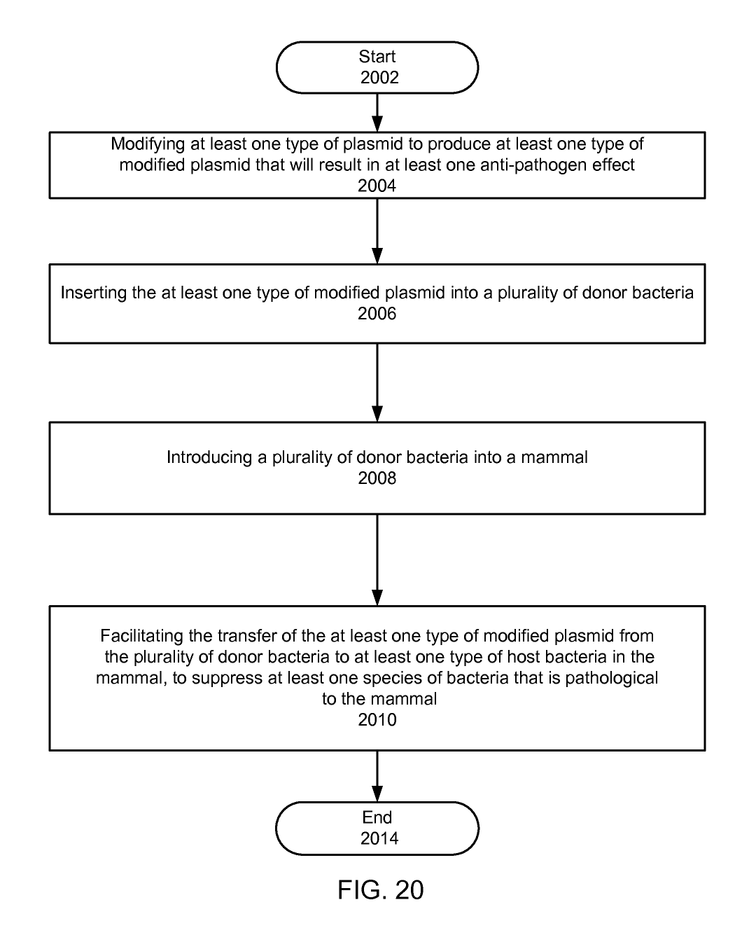

FIG. 20 illustrates a flowchart of a method to use modified plasmids in donor micro-organisms for a beneficial use, in accordance with another embodiment of the invention. The method starts in operation 2002. Operation 2004 is next and includes modifying at least one type of plasmid to produce at least one type of modified plasmid that will result in at least one anti-pathogen effect. Operation 2006 is next and includes inserting the at least one type of modified plasmid into a plurality of donor bacteria. Operation 2008 is next and includes introducing a plurality of the donor bacteria into a mammal. Operation 2010 is next and includes facilitating the transfer of the at least one type of modified plasmid from the plurality of donor bacteria to at least one type of host bacteria in the mammal, to suppress at least one type of species of bacteria that is pathological to the mammal. The method ends in operation 2014.

Since the beginning of the antibiotic era, plasmids from the most quickly adapting bacteria, whether benign or pathogenic, have transferred genes for antibiotic resistance to other bacteria, especially pathogenic bacteria. However, there are at least five strategies to utilize modified plasmids against pathogens and use modified plasmids as direct and immediate substitutes or supplements for antibiotics. One extra advantage of using modified plasmids is that the most promiscuous recipient bacteria at plasmid acquisition that have more quickly and extensively acquired antibiotic resistance can be targeted and more strongly suppressed. An experimental verification of the nearly 100% conjugative transfer within a few hours of an RP4 plasmid conferring resistance to tetracycline from one E. coli donor strain to another tetracycline vulnerable E. coli host strain at human body temperature has demonstrated that a near total conjugative transfer from a donor strain to a host strain can be achieved within a few hours at human body temperature, which is one critical step in showing the feasibility of using a modified RP4 plasmid as a plasmid vehicle for applications against pathogens.

The exemplary embodiments described herein are for purposes of illustration and are not intended to be limiting. Therefore, those skilled in the art will recognize that other embodiments could be practiced without departing from the scope and spirit of the claims set forth below.

What is claimed is:

1. A method of suppressing at least one species of micro-organism that is pathological to an organism, comprising:
modifying at least one type of plasmid to produce at least one type of modified plasmid that will result in at least one anti-pathogen effect;
inserting the at least one type of modified plasmid into a plurality of donor micro-organisms;
increasing the plurality of donor micro-organisms in number by incubation of the plurality of donor micro-organisms;
extracting a plurality of donor micro-organisms containing a plurality of modified plasmids; and
packaging the plurality of donor micro-organisms containing the modified plasmids for a beneficial use.

2. The method of claim 1, further comprising:
introducing a plurality of donor micro-organisms into an organism; and
facilitating the transfer of at least one type of modified plasmid from the plurality of donor micro-organisms to at least one type of host micro-organism in the organism, to suppress at least one species of micro-organism that is pathological to the organism.

3. The method of claim 2, wherein the at least one type of micro-organism includes a bacterium and the at least one type of host micro-organism is also pathological to the organism.

4. The method of claim 2, wherein after facilitating the transfer of the at least one type of modified plasmid from the plurality of donor micro-organisms to at least one type of host micro-organism having a genotype in an organism, the at least one species of micro-organism that is pathological to the organism is suppressed when the at least one type of modified plasmid transfers one or more genes carrying one or more metabolic defects into the genotype of the host micro-organism.

5. The method of claim 2, wherein after facilitating the transfer of the at least one type of modified plasmid from the plurality of donor micro-organisms to at least one type of host micro-organism having a genotype in an organism, the at least one species of micro-organism that is pathological to the organism is suppressed when the at least one type of modified plasmid transfers one or more genes carrying one or more metabolic defects into the genotype of the host micro-organism, such as a defective coding for cell wall peptidoglycan synthesis.

6. The method of claim 2, wherein after facilitating the transfer of the at least one type of modified plasmid from the plurality of donor micro-organisms to at least one type of host micro-organism having a genotype in an organism, the at least one species of micro-organism that is pathological to the organism is suppressed when the at least one type of modified plasmid transfers one or more genes carrying one or more metabolic defects into the genotype of the host micro-organism, such as a defective coding for making a microbial secretion system.

7. The method of claim 2, wherein after facilitating the transfer of the at least one type of modified plasmid from the plurality of donor micro-organisms to at least one type of host micro-organism having a genotype in an organism, the at least one species of micro-organism that is pathological to the organism is suppressed when the at least one type of modified plasmid transfers one or more genes that make the at least one type of host micro-organism an easier target for attack by the innate immune system of the organism and/or an adaptive immune system of the organism.

8. The method of claim 2, wherein after facilitating the transfer of the at least one type of modified plasmid from the plurality of donor micro-organisms to at least one type of host micro-organism having a genotype in an organism, the suppression of at least one species of micro-organism that is pathological to the organism includes making the at least one species of micro-organism an easier target for attack by an innate immune system of the organism and/or an adaptive immune system of the organism by using one or more techniques selected from the group of techniques consisting of:
introducing one or more modified plasmids that encode for one or more proteins that self-opsonize the host micro-organism, making it easier for phagocytosis of the at least one type of micro-organism by a plurality of neutrophils or macrophages of the organism;
introducing one or more modified plasmids that encode for protein production that would more quickly or more strongly activate an innate immune response of the organism by creating and secreting one or more specific cytokines for chemotaxis and/or activating a plurality of phagocytes, wherein the one or more specific cytokines include one or more interleukins; and
activating an adaptive immune response of the organism, either by creating one or more types of more recognizable antigens, or by creating one or more antigens similar to other already recognized antigens that would activate an already existing plurality of memory T cells and memory B cells in the organism resulting from a previously experienced infection of the organism.

9. The method of claim 2, wherein after facilitating the transfer of the at least one type of modified plasmid from the plurality of donor micro-organisms to at least one type of host micro-organism having a genotype in an organism, the suppression of at least one species of micro-organism that is pathological to the organism includes making the at least one species of micro-organism an easier target for attack by an innate immune system of the organism and/or an adaptive immune system of the organism by using one or more techniques selected from the group of techniques consisting of:
interfering with one or more characteristics that help the at least one type of micro-organism in colonization in the organism, such as the formation of a biofilm;
encoding the production of at least one protein that would disrupt a vital metabolic function of the at least one type of micro-organism; and
creating an overwhelming metabolic load in energy or material consumption for the at least one type of micro-organism.

10. A method of suppressing at least one species of micro-organism that is pathological to a mammal, comprising:
modifying at least one type of plasmid to produce at least one anti-pathogen effect;
inserting the at least one type of plasmid into a plurality of donor micro-organisms;
increasing the plurality of donor micro-organisms in number by incubation of the plurality of donor micro-organisms;
extracting a plurality of donor micro-organisms containing a plurality of modified plasmids; and
packaging the plurality of donor micro-organisms containing the modified plasmids for a beneficial use.

11. The method of claim 10, further comprising:
introducing a plurality of donor micro-organisms into a mammal; and
facilitating the transfer of the at least one type of plasmid from the plurality of donor micro-organisms to at least one type of host micro-organism in the mammal, to suppress at least one species of micro-organism that is pathological to the mammal.

12. The method of claim 11, wherein the at least one type of micro-organism includes a bacterium and the at least one type of host micro-organism is also the at least one species of micro-organism that is pathological to the mammal.

13. The method of claim 11, wherein after facilitating the transfer of the at least one type of modified plasmid from the plurality of donor micro-organisms to at least one type of host micro-organism having a genotype in a mammal, the at least one species of micro-organism that is pathological to the mammal is suppressed when the at least one type of modified plasmid transfers one or more genes carrying one or more metabolic defects into the genotype of the host micro-organism.

14. The method of claim 11, wherein after facilitating the transfer of the at least one type of modified plasmid from the plurality of donor micro-organisms to at least one type of host micro-organism having a genotype in a mammal, the at least one species of micro-organism that is pathological to the mammal is suppressed when the at least one type of modified plasmid transfers one or more genes that make the at least one type of host micro-organism an easier target for an innate immune system of the mammal and/or an adaptive immune system of the mammal.

15. The method of claim 11, wherein after facilitating the transfer of the at least one type of modified plasmid from the plurality of donor micro-organisms to at least one type of host micro-organism having a genotype in a mammal, the suppression of at least one species of micro-organism that is pathological to the mammal includes making the at least one species of micro-organism an easier target for an innate immune system and/or an adaptive immune system of the mammal to attack by using one or more techniques selected from the group of techniques consisting of:
introducing one or more modified plasmids that encode for one or more proteins that self-opsonize the host micro-organism, making it easier for phagocytosis of the at least one type of micro-organism by a plurality of neutrophils or macrophages inside the mammal;
introducing one or more modified plasmids that encode for protein production that would more quickly or more strongly activate an innate immune system response by creating and secreting specific cytokines for chemotaxis and/or activating a plurality of phagocytes, including one or more interleukins;
activating an adaptive immune response of the mammal, either by creating one or more already more recognizable antigens, or by creating one or more antigens similar to one or more other common antigens that would activate an already existing plurality of memory T cells and memory B cells in the organism resulting from a previously experienced infection;
interfering with one or more characteristics that help the at least one type of micro-organism in colonization in the mammal, such as the formation of a biofilm;
encoding the production of at least one protein that would disrupt at least one vital metabolic function of the at least one type of micro-organism; and
creating an overwhelming metabolic load in energy or material consumption for the at least one type of micro-organism.

16. A method of suppressing at least one species of bacteria that is pathological to a mammal, comprising:
modifying at least one type of plasmid to produce at least one anti-pathogen effect;
inserting the at least one type of plasmid into a plurality of donor bacteria;
introducing a plurality of donor bacteria into a mammal; and
facilitating the transfer of the at least one type of plasmid from the plurality of donor bacteria to at least one type of host bacteria in a mammal, to suppress at least one species of bacteria that is pathological to the mammal.

17. The method of claim 16, wherein after facilitating the transfer of the transfer of at least one type of modified plasmid from the plurality of donor bacteria to at least one type of host bacteria having a genotype in a mammal, the at least one species of bacteria that is pathological to the mammal is suppressed when the at least one type of modified plasmid transfers one or more genes carrying one or more metabolic defects into the genotype of the host bacteria.

18. The method of claim 16, wherein after facilitating the conjugation of the at least one type of modified plasmid from the plurality of donor bacteria to at least one type of host bacteria having a genotype in a mammal, the at least one species of bacteria that is pathological to the mammal is suppressed when the at least one type of modified plasmid transfers one or more genes that make the at least one type of host bacteria an easier target for an innate immune response of the mammal and/or an adaptive immune system of the mammal.

19. The method of claim 16, wherein after facilitating the transfer of the at least one type of modified plasmid from the plurality of donor bacteria to at least one type of host bacteria having a genotype in a mammal, the suppression of at least one species of bacteria that is pathological to the mammal includes making the at least one species of bacteria an easier target for an innate immune system of the mammal and/or an adaptive immune system of the mammal to attack by using one or more techniques selected from the group of techniques consisting of:
introducing one or more modified plasmids that encode for one or more proteins that self-opsonize the host bacteria, making it easier for phagocytosis of the at least one type of bacteria by a plurality of neutrophils or macrophages;
introducing one or more modified plasmids that encode for protein production that would more quickly or more strongly activate an innate immune response by creating and secreting one or more specific cytokines for chemotaxis and/or activating a plurality of phagocytes, wherein the one or more specific cytokines include one or more interleukins; and
activating the adaptive immune response, either by creating one or more recognizable antigens, or by creating one or more antigens similar to other common antigens that would activate an already existing plurality of memory T cells and memory B cells in the mammal resulting from a previously experienced infection.

20. The method of claim 16, wherein after facilitating the transfer of the at least one type of modified plasmid from the plurality of donor bacteria to at least one type of host bacteria having a genotype in a mammal, the suppression of at least one species of bacteria that is pathological to the mammal includes making the at least one species of bacteria an easier target for attack by an innate immune system of the mammal and/or an adaptive immune system of the mammal by using one or more techniques selected from the group of techniques consisting of:
interfering with one or more characteristics that help the at least one type of bacteria in colonization in the mammal, such as the formation of a biofilm;
encoding the production of at least one protein that would disrupt at least one vital metabolic function of the at least one type of bacteria; and
creating an overwhelming metabolic load in energy or material consumption for the at least one type of bacteria.
